(19) United States
(12) Patent Application Publication
(10) Pub. No.: US 2021/0236380 A1
(43) Pub. Date: Aug. 5, 2021

(54) ENTERTAINMENT SYSTEM INCLUDING A SEX APPARATUS WHICH CAN BE REMOTELY CONTROLLED BY MEANS OF A DEVICE WITH ACCESS TO INTERNET

(71) Applicant: Álvaro Antonio ZAMORANO SILVA, Mexico City (MX)

(72) Inventor: Álvaro Antonio ZAMORANO SILVA, Mexico City (MX)

(21) Appl. No.: 16/635,544

(22) PCT Filed: Dec. 8, 2017
(86) PCT No.: PCT/IB2017/057756

§ 371 Date: Mar. 25, 2021

(30) Foreign Application Priority Data
Jul. 31, 2017 (MX) MX/A/2017/009915

(51) Int. Cl. A61H 19/00; G08C 17/02; H04W 76/10; A61H 23/02; G05B 15/02; B25J 9/16

(52) U.S. Cl. CPC A61H 19/00; G08C 17/02; H04W 76/10; A61H 23/0254; G05B 15/02; G16H 40/63; A61H 2201/5043; A61H 2201/1669; A61H 2201/5012; A61H 19/44; A61H 2201/0134; B25J 9/1689

(57) ABSTRACT

An entertainment system for adults is described and shown, where control signals can be one-way and remotely transmitted, by means of a mobile device of a used called subscriber, to a mobile device which in turn retransmits the control signals to a sexual apparatus used by a user called provider in the course of a video call that is managed by a data server while audio and/or video signals are transmitted from the provider mobile device to the subscriber mobile device via a streaming server.

### CROSS-REFERENCE TO RELATED APPLICATIONS

This application claims priority to MX Patent Application No. MX/a/2017/009915 filed on Jul. 31, 2017, and to PCT Application No. PCT/IB2017/057756 filed on Dec. 8, 2017, the entire contents of which are hereby incorporated by reference.

### FIELD OF THE INVENTION

The invention relates to an entertainment system, particularly for adults, comprising at least one device of the type known as a sex toy for stimulating the erogenous zones of a user identified as “provider”, for example a woman, wherein each sexual apparatus is capable of being remotely controlled by another user identified as “subscriber” during the course of a video call through the use of devices having access to a telecommunications network, for example, the Internet, such as personal computers, smartphones, tablets, and the like or equivalent devices.

The invention also relates to a sexual apparatus that is configured to communicate with a “provider device”, and wherein the sexual apparatus is further configured to be activated at different speeds or intensities according to a selection made by the “subscriber” during the course of a video call between the respective “provider device” and “subscriber device”, which video call is managed by the system.

The invention further relates to methods for transmitting both audio and/or video data and control data, in real time, between the respective “provider device” and the “subscriber device” during the course of a video call managed by the system.

### BACKGROUND OF THE INVENTION

There is nowadays a wide variety of devices on the market for stimulation of erogenous zones of both men and women, which are commonly called “sex toys”, for example, artificial vaginas, mainly for use by men, and dildos, mainly for use by women. Such sex toys may even have mechanical and/or electronic capabilities to enhance sexual stimulation.

Devices and systems with improved functionalities have recently been developed that allow virtual interaction between the users of said sex toys through the use of telecommunications networks, mainly the Internet, in such a way that it is possible to simulate or replicate the signals detected by a sex toy that is used, for example, by a man, in another sex toy that is used, for example, by a woman, and where the respective sex toys are remotely located.

For instance, from DE 10 2009 014 044 A1 a system comprising a network server, and a plurality of devices comprising actuators and sensors for recording, transmission and reproduction of signals between said devices connected remotely via Internet is known. The device is configured to detect signals which are transmitted to another remote device, and to produce stimulation based on signals received from the other remote device. The disclosed system thus consists in a bidirectional-signals detection and transmission system because the signals detected by one sex toy are transmitted to the other sex toy and vice versa, to reflect the sensations of a sexual act in such a way realistic as possible.

Similarly, a system having a sex toy for a man and a sex toy for a woman is known from WO 2014/127522 A1 and WO 2014/127523 A1. In this known system, each sex toy is linked to a respective control terminal to allow the reception and transmission of signals between the two sex toys, thus enabling interaction between users.

A system having multiple sex toys is known, for example, from CA 2 785 083 and US 2004/0082831 A1, wherein each user utilizes a sex toy that detects signals by means of sensors, said signals are then transmitted to another sex toy, and which also receives signals from said another sex toy, thus allowing interaction between several users through the exchange of control signals between two or more users.

Another similar system is the one disclosed in DE 10 2012 200 252 A1, where a user has a sex toy and a mobile device, such as a telephone, by means of which he can connect with the sex toy and mobile device, such as a telephone, of another user for sending and receiving signals detected by sensors arranged in each sex toy, thus providing two-way control via an Internet connection through a server.

In the state of the art, therefore, there are known sex toys and systems intended to provide a virtual interaction among two or more users through the exchange of control signals detected by sensors arranged in the sex toys. In other words, the known systems are intended to provide bidirectional control between sex toys based on the signals detected by the sensors of each sex toy.

Nevertheless, known sex toys have limited or no functions in terms of performing reciprocating movements requiring no direct manipulation by users and, furthermore, in known systems the possibilities of providing real time and safe interaction between a user remotely controlling the movements of the sex toy that is used by another person during the course of a video call have not been exploited.

### BRIEF DESCRIPTION OF THE INVENTION

Therefore, the invention is based on the object of providing a system and sexual apparatus that surpass the capabilities and overcome disadvantages of the prior art.

In an aspect of the invention, a sexual apparatus is provided comprising a reciprocating motion mechanism including at least one electric motor and a plurality of links that are configured to convert the rotary motion of the electric motor into a reciprocating motion along a substantially straight axis. A sex toy, such as a dildo, can be attached to an end link of the plurality of links. In this way, the sexual apparatus has the ability of generating an essentially linear reciprocating movement of the sex toy (dildo) in function of the rotational speed of the electric motor.

With the aim to control the rotational speed of the electric motor, the sexual apparatus further comprises a control and communication unit including a central processing unit and an integrated circuit board in communication with the central processing unit by means of an interface, wherein the control and communication unit is powered by at least one power source. The control and communication unit has the capability of connecting to at least a wired network, for example a local network via an Ethernet cable, and/or a wireless network, for example a local network via Wi-Fi, Bluetooth or infrared. In this way, the sexual apparatus is able to communicate with a telecommunications network, such as the Internet, and in this way to receive control signals generated by a mobile device, such as a cell phone, personal computer, portable computer, tablet, etc., through a server having Internet connection.

In a further aspect of the invention, the central processing unit can comprise, or it can be connected with, a display, and a keyboard, allowing visualization and input of data, in such a way that a person can configure a connection mode of the sexual apparatus with a telecommunications network. Alternatively, the display may consist in a touch screen including a keyboard displayed thereon.

In a further aspect of the invention, the sexual apparatus comprises a base and a housing supporting and protecting the communication and control unit and the reciprocating mechanism. In addition, with the aim to provide a sexual apparatus that can be easily manipulated due to the light weight thereof, the base is a base having a hollow interior that can be filled, through a removable plug, for example, with a liquid such as water, or with a flowing solid substance, such as sand, in particular silica sand. By providing a fillable hollow base, the sexual apparatus can be sold with said base empty to allow easy and comfortable handling of the apparatus by the person purchasing the apparatus.

In a further aspect of the invention, the sexual apparatus comprises a structure for adjusting the tilt and/or position of the reciprocating mechanism relative to the base. In this way, the person using the sexual apparatus can, on the one hand, modify the distance between the reciprocating mechanism and the base, in particular move the reciprocating mechanism closer to or away from the base, as well as modify the relative angle between the displacement axis of the sex toy (dildo) and the sexual apparatus base, and on the other hand, improve comfort and reduce the risk of injury to the person using the device.

In a further aspect of the invention, at least one end link, that is to say a free end link, of the reciprocating mechanism comprises a connector, in particular of the type known as fast connector. Furthermore, the sexual apparatus comprises a sex toy, of the type known as dildo. The sex toy (dildo) also includes a fast connector. For example, the reciprocating mechanism connector is a male connector, and the sex toy connector is a female connector. Alternatively, the reciprocating mechanism comprises a female fast connector, and the sex toy comprises a male fast connector. In this way, the sex toy can be easily, safely and removably connected to the reciprocating mechanism of the sexual apparatus.

In a further aspect of the invention, the sexual apparatus can comprise a holder for placing a mobile device. This has the advantage that the sexual apparatus and the mobile device are easy to manipulate during use of the sexual apparatus.

In a further aspect of the invention, a system comprising at least one data server and at least one streaming server is provided, which servers are configured to connect to a telecommunications network, such as the Internet. The system further comprises a plurality of mobile devices, such as smartphones, tablets, computers, and the like or equivalent devices, which are intended to be used by respective subscribers and providers previously registered in the system, by generating a user account. In a broad sense, the mobile devices have the capability to connect to a telecommunications network, such as the Internet, as it is known in the art, for example, through a GSM/GPRS/3G/4G module communicating with a base station which in turn communicates with the telecommunications network (Internet), and/or through a wireless network module communicating with an access port, such as a router, which in turn communicates with the telecommunications network (Internet). In addition, the system comprises a plurality of sexual apparatuses intended to be used by respective providers. Each mobile device is configured to connect to a telecommunications network, such as the Internet. Furthermore, it is intended that at least the provider mobile devices, but also the subscriber mobile devices, comprise at least one camera configured for transmission and/or recording of video data and at least one microphone configured for transmission and/or recording audio data. In this way, the mobile devices are configured to send and/or receive data signals that may include audio and/or video, as well as control signals, through the telecommunications network (Internet), as it is known in the art. In this way, subscribers and providers, who are previously registered in the system, can make use of the system for transmitting audio/video signals, at least from the providers, and control signals, from the subscribers, during the course of a video call that is managed by the data server through the telecommunications network (Internet).

In a further aspect of the invention, the data server is configured to manage the registration of users, in particular as subscribers and as providers, as well as to provide an interactive (digital) platform allowing the transmission of control signals from the subscriber mobile devices to the provider mobile devices and the latter in turn to the provider sexual apparatuses, while the streaming server is configured to manage the transmission of audio and/or video signals from the provider mobile devices to the subscriber mobile devices. This has the advantage that the audio and/or video signals, which commonly require a large amount of information, are transmitted separately from the control signals.

In a further aspect of the invention, the data server and the streaming server are two independent or separate entities communicating with each other. This has the advantage that each server can be located remotely with respect to the other server. Alternatively, the data server and the streaming server are part of the same equipment that integrates both components, in such a way that they can be at the same location.

In a further aspect of the invention, each sexual apparatus of the system is adapted to connect to the telecommunications network (Internet) following a previous configuration by means of the provider mobile device. In this sense, the control and communication unit of the sexual apparatus is configured to communicate with an access port, such as a router or a local modem, which in turn has a connection with the telecommunications network (Internet), as it is known in the art. This has the advantage that every time the provider desires to login in the system, the connection between the sexual apparatus and the telecommunications network (Internet) can be easily and quickly set up, and thereby it is enabled to receive control signals generated by a subscriber mobile device registered in the system.

In an additional aspect of the invention, once the connection between the provider sexual apparatus and the telecommunications network (Internet) has been configured, the provider who was previously registered in the system through a user registration can login in the system and can be shown in a providers list or “pool” which is managed by the data server. The providers list or “pool” shall include all registered and available providers, that is who they are currently connected (logged in) in the system, and it will be visible to a plurality of subscribers registered and connected (logged in) in the system. In this sense, each subscriber previously registered can access the system by means of a respective mobile device and select from the providers list or “pool” at least one provider to establish a video call between their devices. Following the selection of at least one provider, the subscriber can generate and transmit, through his mobile device, a connection request signal with that selected provider. Subsequently, the provider mobile device displays a connection request indication. The provider can choose to accept or to reject the incoming connection request. In case of rejecting the request, the system notifies the subscriber about the provider's decision so that the subscriber can select another provider available from the providers list or “pool” and send a new connection request. In case the request is accepted, the system performs a step to determine whether the subscriber can effectively make the connection with the provider, for example whether the subscriber's web browser is compatible to make the video call. Subsequently, the system, and in particular the data server, enables a connection between the subscriber device and the provider device. During this connection, the subscriber mobile device can generate control signals which are transmitted, via the data server, to the provider mobile device and the latter in turn relays them to the sexual apparatus; simultaneously, the provider mobile device, in particular the camera and microphone, transmits audio and/or video signals, via the streaming server, to the subscriber mobile device. The control signals generated on the subscriber's side are intended to control the rotational speed of the motor and, as a consequence, the reciprocating movements of the sex toy (dildo), of the sexual apparatus. In turn, the audio and/or video signals generated by the provider mobile device are received on the subscriber mobile device, thus establishing a so-called “video call”. In particular, a one-way control connection is established between a subscriber and a provider, as well as a one-way video call between them. This has the advantage that the subscriber only transmits control signals to the provider mobile device, and the provider only transmits audio and/or video signals to the subscriber, thus creating a system that allows to protect the subscribers identity, since they do not transmit audio and/or video signals.

Although during the one-way video call described above the provider mobile device only transmits audio and/or video signals, it is also possible, in a further aspect of the invention, that the subscriber mobile device transmits audio and/or video signals to the provider mobile device. In this way, a two-way video call can be established between the mobile devices of the subscriber and provider. Furthermore, given that the transmission of audio and/or video signals generally involves a large volume of data, it is intended that this stage is managed by the streaming server.

In a further aspect of the invention, the data server manages the video call, that is, the beginning and finalization of audio and/or video signals transmission. However, the actual transmission of audio and/or video signals is carried out via the streaming server. Accordingly, the data traffic of both the data server and the streaming server is reduced.

Therefore, the present invention provides a system where control signals can be remotely and one-way transmitted, by a mobile device of a user called subscriber, to a mobile device which in turn relays the control signals to a sexual apparatus used by a user called provider in the course of a video call, which is managed by a data server while audio and/or video signals are transmitted from the provider mobile device to the subscriber mobile device via a streaming server.

Additional features and advantages of the sexual apparatus and system according to the invention will become apparent from the following detailed description in conjunction with the attached drawings, which show in an exemplary non-limiting manner embodiments of the invention. The drawings show:

### BRIEF DESCRIPTION OF THE DRAWINGS

FIG. 1 a schematic representation of a sexual apparatus according to an embodiment of the present invention;

FIG. 2 a schematic representation of an entertainment system for adults according to an embodiment of the present invention; and

FIG. 3 a flow chart of a method for establishing a video call connection between a provider and a subscriber and for enabling the transmission of control signals to a sexual apparatus, according to an embodiment of the present invention.

### DETAILED DESCRIPTION OF THE INVENTION

In the sense of the present invention, the term “provider” refers to a user owning and using a sexual apparatus for stimulation of his/hers erogenous zones, and who has registered and logged-in in the system according to the invention. Also, the term “subscriber” refers to a user who has registered and logged-in in the system, and who can remotely generate and transmit control signals to a provider sexual apparatus in the course of a video call.

On the other hand, the terms “provider toy”, “toy device” and “sexual apparatus” will be used interchangeably, unless otherwise indicated, and they refer to the apparatus with which the erogenous zones of a person, in particular a woman or in the sense of the present invention the “provider”, are stimulated with, and who has the ability to perform a reciprocating movement and to connect to a telecommunications network to receive control signals to change the speed and/or intensity of the reciprocating movement.

Sexual Apparatus

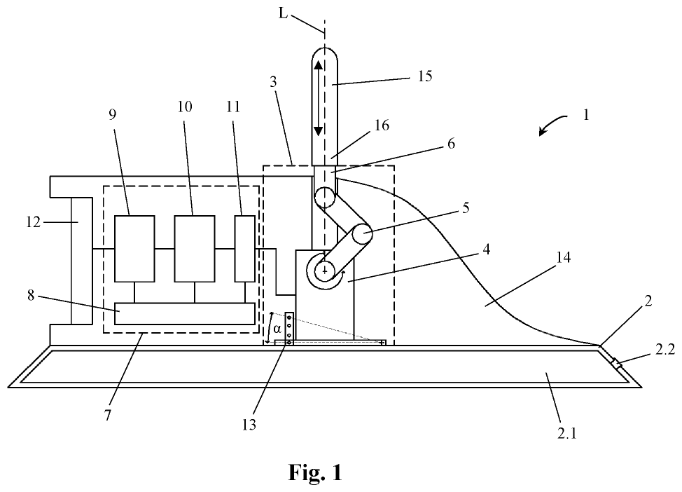

With reference now to FIG. 1, the sexual apparatus 1 according to an embodiment of the present invention is schematically shown. The sexual apparatus 1 comprises a base or platform 2 to which a reciprocating motion mechanism 3 is attached, a control and communication unit 7, a housing 14 designed to protect the components of the sexual apparatus 1, and a sex toy 15 of the type known as a dildo.

The reciprocating motion mechanism 3 comprises at least one electric motor 4, in particular an electric stepper motor, and a plurality of links 5 configured to convert the rotary movement of the motor 4 into a reciprocating movement along a substantially straight axis L.

The control and communication unit 7 is configured to control the rotational speed of the electric motor 4. The control and communication unit 7 comprises a central processing unit 9 and an integrated circuit board 11 in communication with the central processing unit 9 by means of an interface 10. Here, the elements of the control and communication unit 7 are powered by at least one power source 8, which may comprise, for example, a battery source, or it may consist in a conventional 127 V household electrical connector with its respective alternating current to direct current (AC/DC) transformer circuit.

By way of example, the central processing unit 9 consists in a computing module having the capability of processing logical information and establishing a connection with a telecommunication network, such as a local network or even the Internet, as it is known by those skilled in the art. According to an embodiment of the invention, the central processing unit 9 is, for example, a Raspberry PI®, Model 3 computer, which is commercially available in the market at a low cost and is characterized by being compact and programmable in a simple way and free to use.

Additionally, the central processing unit 9 comprises or is connected to a display 12, in particular a touch screen, which includes a keyboard for displaying and inputting data. In use, the keyboard including display 12 can be manipulated by a person to configure a connection mode between the control and communication unit 7, in particular the central processing unit 9, and a telecommunications network, such as the Internet.

Although it is not shown in the drawings, in an alternative embodiment of the invention, the display 12 and a separate peripheral keyboard can be connected to the central processing unit 9 to enable the connection setup between the central processing unit 9 and the telecommunications network (Internet) instead of the touch screen described above.

Continuing with FIG. 1, the base 2 comprises a hollow inner space 2.1 that is designed to be filled, for example, with a liquid such as water, or with a flowing solid substance such as sand, through a plug 2.2. According to these features, the base 2 of the apparatus 1 is provided, for its public sale, as an empty element which substantially reduces the weight of the apparatus thus allowing easy and comfortable handling.

Additionally, as it can be observed in FIG. 1, a connector 6 is arranged in an end link of the plurality of links 5 of the reciprocating mechanism 3. Said connector 6 is configured to be removably connected to a connector 16 of a sex toy 15, such as a dildo. According to one embodiment of the invention, the connector 6 consists in a male fast connector, and connector 16 consists in a female fast connector, both of which are known in the market. Alternatively, according to another embodiment of the invention, connector 6 consists in a female fast connector, and connector 16 consists of a male fast connector. Therefore, the connection between the connectors 6 and 16 can be made easily and safely, and it also allows for separation of the connectors for removal of the sexual apparatus from the apparatus 1.

According to an embodiment of the invention, the mechanism 3 additionally comprises a structure 13 allowing for adjustment of the position and/or tilt of the mechanism 3 relative to the base 2, for example, to move said mechanism 3 away from or closer to the base 2 and/or to change the tilt angle α of the mechanism 3 relative to said base 2.

As mentioned at the beginning, the central processing unit 9 is configured to establish a connection with a telecommunications network to receive rotational speed control signals of the electric motor 4. In particular, the connection between the central processing unit 9 of the sexual apparatus 1 and the telecommunications network can be done through a local network, for example, a wide area network (WAN) or the like. This configuration has the advantage that the connection between the sexual apparatus 1 and the local network can be carried out in a way that does not require an Internet connection.

The integrated circuit board 11 consists in an electronic circuit transforming and/or adapting electrical signals coming from the central processing unit 9 into electrical pulses that control the rotational speed of the electric motor 4. However, the signal transmission between the central processing unit 9 and the integrated circuit board 11 is made via an interface 10. The interface 10 consists, for example, in an Arduino® circuit, which is known in the market, and constitutes a controller circuit (driver) of the electric motor 4.

Entertainment System for Adults

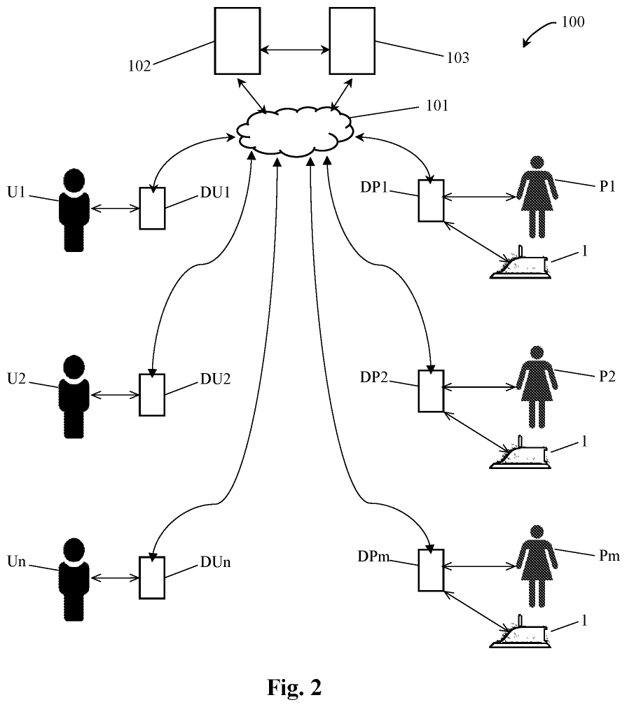

Reference will now be made to FIG. 2, which schematically shows the elements of the entertainment system 100 for adults in accordance with an embodiment of the invention.

System 100 comprises a data server 102 and a streaming server 103, both of which are configured to connect to a telecommunications network 101, such as the Internet; a plurality of mobile devices (DP1, DP2, . . . , DPm) and a plurality of sexual apparatuses 1 belonging to a plurality of providers (P1, P2, . . . , Pm), respectively; and a plurality of mobile devices (DU1, DU2, . . . , DUn) belonging to a plurality of subscribers (U1, U2, . . . , Un), respectively.

According to an embodiment of the invention, the server 102 and the streaming server 103 can be configured in such a way that they are both integrated into the same equipment. Alternatively, and advantageously, it is intended that the server 102 and the streaming server 103 are configured or installed independently of each other, which avoids the delay in data transmission because it reduces the information transmission load.

In the sense of the present invention, it is intended that each sexual apparatus 1 (see FIG. 1) is acquired by a plurality of providers (P1, P2, . . . , Pm), for example, in each case by a woman who desires to provide the entertainment service through the use of system 100. In addition, it is intended that each provider (P1, P2, . . . , Pm) has a mobile device (DP1, DP2, . . . , DPm) such as a mobile phone, a tablet, a personal computer or similar device including, at least, a video camera, a microphone, a speaker, a network connection module, and an Internet browser, for example Google Chrome® or Firefox® browser, so as to be compatible with the system 100 functionalities. On the other hand, it is intended that a plurality of subscribers (U1, U2, . . . , DUn) can use the system 100 by means of a respective mobile device (DU1, DU2, . . . , DUn), for example, a mobile phone, tablet, personal computer or similar device comprising, at least, a microphone, a speaker, an Internet connection module and an Internet browser, for example, Google Chrome® or Firefox® browser, so as to be compatible with the system 100 functionalities.

As mentioned above, each one of the subscribers (U1, U2, . . . , Un) and each one of the providers (P1, P2, . . . , Pm) can access the system 100 by means of a previous “user registration”. This has the function of guaranteeing the safe use of the system since each person must accept the use terms and conditions as well as demonstrate legal age to use the system. This registration stage is managed by the respective subscriber and provider mobile devices through the data server 102 by establishing at least a username and a password. Particularly, when a user desires to have the functionality of remotely controlling a sexual apparatus, said user generates a registration as “subscriber”. Alternatively, when a user desires to use the sexual apparatus and allow its remote control, said user generates his/her registration in the system as “provider”. The data server 102 includes a database, which is isolated from the Internet for security reasons, and which includes all information related to user registrations.

Having generated a “user registration” by the providers (P1, P2, . . . , Pm), they can establish the connection settings of their respective sexual apparatuses 1, as described below.

Setup of the Sexual Apparatus in the System

As discussed in the brief description of the invention, the sexual apparatus 1 can be remotely controlled by receiving control signals. However, to enable this function of the apparatus 1, it must be configured by the provider according to the following sequence:
The provider (P1, P2, . . . , Pm) who has acquired a sexual apparatus 1, and who has already generated his “user registration” in the system 100, accesses the system through her/his mobile device (DP1, DP2, . . . , DPm) and turns the apparatus 1 on.
Once the apparatus 1 is turned on, the central processing unit 9 (see FIG. 1) performs a scan for available wireless connections within a predetermined range, and shows a list of those available wireless connections on the display 12 thus allowing the provider (P1, P2, . . . , Pm) to select one of these.
The provider (P1, P2, . . . , Pm) can then select a network to be used and, in response, the display 12 will show a window in which it is possible to enter, through the keyboard, the user's data and password pertaining to the selected network; for example, the network may be a local wireless network enabled by a wireless router or modem, which in turn is connected, wired or wirelessly, to a telecommunications network, such as the Internet.
Once the connection is established, that is to say when a “successful connection” is assured, an IP address is shown on the display 12 of the apparatus 1, which must be entered in the mobile device (DP1, DP2, . . . , DPm) of the provider (P1, P2, . . . , Pm) The provider (P1, P2, . . . , Pm) then directs to a menu that is displayed on his/her mobile device (DP1, DP2, . . . , DPm) and that includes a section called “DEVICE” in which the IP address is required to be input, which is shown on the display 12 of his/her apparatus 1.

Once the IP address of the apparatus 1 is entered, the mobile device (DP1, DP2, . . . , DPm) sends a connection test command between the latter and the apparatus 1, which indicates that the connection has been made successfully. At this point, it is now possible to use the apparatus 1 in the system 100, since a connection has already been established between the apparatus 1 and the mobile device (DP1, DP2, . . . , DPm) of the provider (P1, P2, . . . , Pm).

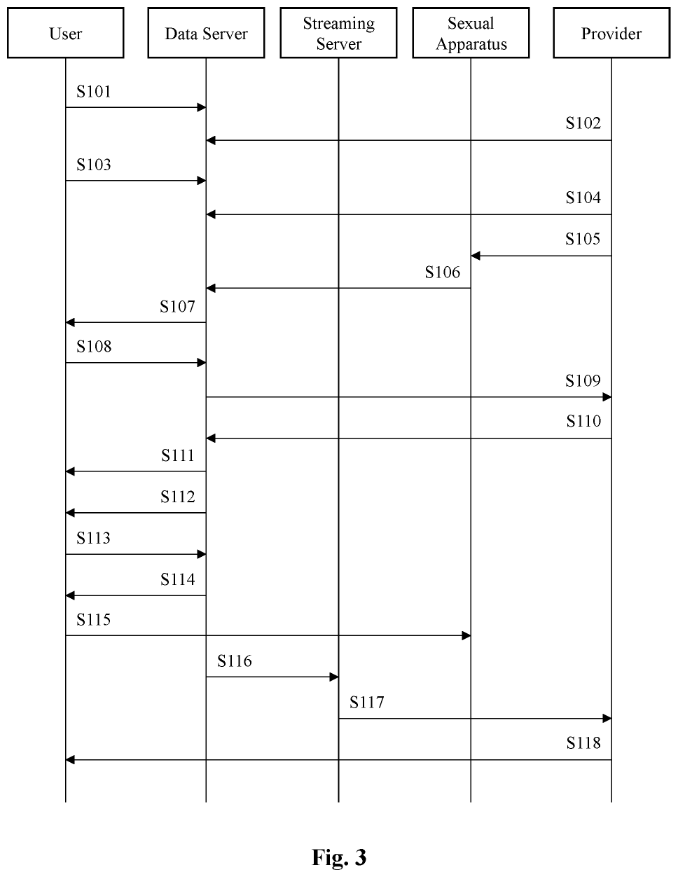

Once the providers (P1, P2, . . . , Pm) have accessed the system 100 and configured the connection of their respective sexual apparatuses 1, and the subscribers (U1, U2, . . . , Un) have accessed the system 100, the data server 102 can manage data transmission between providers (P1, P2, . . . , Pm) and subscribers (U1, U2, . . . , Un) according to a data transmission management method, which is illustrated in FIG. 3 and which is described in detail below in conjunction with FIG. 2.

Data Transmission Management Method between Providers and Subscribers

Each subscriber and provider must register in the system 100 (FIG. 2) to enable use thereof. For example, a person who wishes to have the functionality of remotely controlling a sexual apparatus and receiving, on his/her mobile device, audio/video signals from another person who owns said sexual apparatus, generates a “user registration” in the system by using his/her Internet browser (for example, Google Chrome® or Firefox®) on his/her respective mobile device by setting a username and password, and selecting an option to register in the system as “subscriber” (step S101).

Alternatively, a person who wishes to be stimulated with the sexual apparatus and transmit, through her/his mobile device, audio/video signals, generates a “user registration” in the system by using her/his Internet browser (for example, Google Chrome® or Firefox®) on her/his respective mobile device by setting a username and password, and selecting an option to register in the system as “provider” (step S102).

Once registered as subscriber or provider, they have access to the system through their respective mobile devices through an Internet connection. This stage is commonly known as “login” in the system, for which each subscriber and each provider input their username and password data (steps S103 and S104, respectively). The data server validates the information and allows or denies access depending on a match or non-match of the information input.

On the provider's side, whom has already acquired the sexual apparatus and has accessed into the system, manipulates (step S105) and configures (step S106) the sexual apparatus, as previously described.

Once the providers' apparatuses have been configured, the system generates and displays (step S107) a “providers pool” or list which is visible, on their respective mobile devices, to the plurality of subscribers logged-in in the system.

On the subscriber's side, by means of their mobile devices, they see the “providers pool” or list and then they can select at least one provider from said list, and after selecting at least one provider, the subscriber generates and transmits to the server (step S108) a “Connection Request” with said provider to initiate a video call during which said subscriber can control the provider's device.

In response, the server generates and transmits (step S109) a “Connection Request” notification. Then, the provider receives a “Connection Request” notification on her/his mobile device, and the provider can then choose an option to “Accept Request” or “Reject Request”. This decision is sent (step S110) to the data server.

In case the provider chooses “Reject Request”, the system notifies (step S111) the subscriber about the provider's choice, so that the provider can select another provider from the “providers pool” or list and generate another “Connection Request”, namely the method returns to step S108.

In case the provider chooses “Accept Request”, the data server can manage a sub-process to determine whether the subscriber has the capacity to make the connection with the provider, which may include the data server determining (step S112) whether the subscriber's browser is compatible for making the video call.

Once the subscriber verifies (step S113) that he/she can make the connection with the selected provider, then the server establishes (step S114) the connection and manages (step S115) the transmission of control data from the subscriber mobile device to the provider mobile device, and the latter in turn relays the control signals to the provider sexual apparatus. Simultaneously, the data server generates and transmits (step S116) a command to the streaming server so that the latter in turn enables (step S117) the transmission of audio and/or video data from the provider mobile device to the subscriber mobile device (step S118).

With reference to FIGS. 1 to 3, a subscriber (U1, U2, . . . , Un) can generate and send (step S115), from his/her mobile device (DU1, DU2, . . . , DUn), control signals that allow remote control of the rotational speed of the electrical motor 4 of the sexual apparatus 1, during the course of a video call with a provider (P1, P2, . . . , Pm), wherein the video call is managed and/or controlled by a data server 102. Additionally, the data server 102 is in communication with a streaming server 103 which has the function of transmitting (step S118) audio and/or video data from the provider (P1, P2, . . . , Pm) to the subscriber (U1, U2, . . . , Un).

For security reasons, it is not possible to establish a connection between the subscriber mobile device and the sexual apparatus since the sexual apparatus does not possess the privileges necessary to be considered a visible device in the network but it is only configured to be associated with the provider mobile device through the local network; therefore, advantageously, the sexual apparatus does not require an Internet connection.

Alternative embodiments and further modifications will be apparent to those skilled in the art, which should be understood to be within the spirit and scope of the appended claims.

### LIST OF REFERENCE NUMBERS

1: Sexual apparatus
2: Base
2.1: Hollow inner space of base 2
2.2: Base plug 2
3: Reciprocating motion mechanism
4: Electric motor
5: Links
6: Connector
7: Control and communication unit
8: Power supply
9: Central processing unit
10: Interface
11: Integrated circuit board
12: Display
13: Position/tilt adjustment structure
14: Housing
15: Sex toy (dildo)
16: Sex toy connector 16
L: Longitudinal displacement axis

1-20. (canceled)

21. A sexual apparatus comprising:
a base to which a reciprocating motion mechanism is attached to;
a reciprocating motion mechanism attached to the base;
a control and communication unit;
a power source; and
a housing, wherein
the reciprocating motion mechanism comprises an electric motor and a plurality of links configured to convert the rotational motion of the electric motor into a reciprocating motion along a substantially straight axis,
the control and communication unit comprises a central processing unit and an integrated circuit board in communication with the central processing unit by means of an interface, and
the control and communication unit is configured to control the rotational speed of the electric motor in function of at least one control signal, wherein the at least one control signal is received by the control and communication unit through a wired or wireless communication connection with a provider device, wherein said provider device receives the at least one control signal through an Internet connection from a subscriber device.

22. The sexual apparatus according to claim 21, wherein
the control and communication unit communicates with a display, and
the display comprises a keyboard for data display and input.

23. The sexual apparatus according to claim 21, wherein
the base comprises a hollow inner space configured to be filled with a fluid material.

24. The sexual apparatus according to claim 21, further comprising:
a connector at an end link of the plurality of links configured to connect, in a removable manner, to a connector of a sex toy.

25. The sexual apparatus according to claim 21, wherein
the reciprocating motion mechanism further comprises a structure allowing adjustment of the position and/or tilt of the reciprocating motion mechanism relative to the base.

26. The sexual apparatus according to claim 21, wherein
the integrated circuit board consists in an electronic circuit transforming and/or adapting electrical signals from the central processing unit into electrical impulses controlling the rotational speed of the electric motor.

27. The sexual apparatus according to claim 21, wherein
the wired communication connection includes at least one of a wide area network (WAN) and a local network through Ethernet cable to receive the at least one control signal.

28. The sexual apparatus according to claim 21, wherein
the display is configured to establish a connection between the apparatus and a provider mobile device by means of the central processing unit.

29. The sexual apparatus according to claim 27, wherein
the wireless communication connection includes at least one of a Wi-Fi, Bluetooth, infrared network.

30. The sexual apparatus according to claim 21, wherein
the provider device comprises at least one of a cell phone, personal computer, portable computer or tablet.

31. The sexual apparatus according to claim 21, wherein
the subscriber device comprises at least one of a cell phone, personal computer, portable computer or tablet.
